(19) United States
(12) Patent Application Publication
(10) Pub. No.: US 2005/0042751 A1
(43) Pub. Date: Feb. 24, 2005

(54) Generation and use of new types of dendritic cells

(76) Inventors: Michel Goldman, Bruxelles (BE); Jamila Ismaili, Fajara (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/494,537

(22) PCT Filed: Oct. 30, 2002
(86) PCT No.: PCT/EP02/12101

(30) Foreign Application Priority Data
Oct. 31, 2001 (EP) 01870234.0

(51) Int. Cl. C12N 5/08

(52) U.S. Cl. 435/372

(57) ABSTRACT

The present invention relates to a method for the differentiation and/or maturation of immature myeloid dendritic cells (DC) into HLA-DR, CD86, CD83 and IL12 (p40) dendritic cells comprising incubating said DC with dg T cells; and to a method for the differentiation and/or maturation of immature myeloid dendritic cells (DC) into HLA-DR, CD86, CD83 and IL12 (p70) dendritic cells comprising incubating said DC with activated dg T cells. Specific compositions and uses thereof are described.

### TECHNICAL FIELD

The present invention relates to the improvement of the therapy for the treatment or prevention of cancer, infections and autoimmune diseases in particular in the development of new dendritic cells carrying a superior character in inducing T cell responses.

### BACKGROUND ART

For years people try to understand the regulatory system of the immunological response. It is clear that the immune reaction results from a complex interaction between cellular and humoral responses.

Dendritic cells (DC) represent the most important antigen presenting cells for the induction of primary T cell responses (1). In order to efficiently exert their function in lymphoid organs, DC have to undergo a maturation process which is initiated in peripheral tissues. Maturation of DC results in the expression of high levels MHC and costimulatory molecules on their membrane and is often associated with the secretion of interleukin (IL)-12 (2-3), a critical factor for the development of Th1-type responses.

As this cell type is endowed with the unique capacity to cluster naïve T cells, it has been proposed as a natural adjuvant, aiming at the triggering of T-cell responses against poor immunogens, such as tumor-associated Ag (TAA). The realization of clinical trials has long been impaired by the low frequency of circulating DC available in the blood. The development of methods of generating large numbers of DC from hemapoietic precursors has recently allowed the initiation of pioneer clinical trials. Human myeloid DC can be easily generated in vitro by culturing monocytes in presence of GM-CSF and IL-4 whereas the so-called lymphoid DC have been obtained by isolation of precursors from blood or lymph nodes. Recently, the inventors described a new type of stable dendritic cell (DC) which is more mature and is more potent in activating the immune system compared to other known stable DCs (EP00870273.0). Indeed, monocytes cultured in IL3 and IFN-β differentiate into IL3-R+ CD11c+ myeloid dendritic cells with potent T cell stimulatory activities.

The GM-CSF/IL4 DC have been used in clinical trials in cell therapy. However, using these conditions, only 67% of immunized patients showed an increase in response to the treatment. Therefore it is important to improve the method, conditions or substances to improve the efficacity of the treatment.

### OBJECTS OF THE INVENTION

The present invention is directed towards providing a method for the production of superiour dendritic cells which can be used in cell therapy to eliminate or prevent more efficiently the deleterious effects of invasive cells in patients.

In contrast to the well studied influence of cytokines on the DC maturation, less attention has been paid so far to the possible contribution of cells taking part in innate immune defenses to this effect; The inventors found surprisingly that dendritic cells, present in an early developing stage (referred to as immature myeloid DCs in this patent application) could be further differentiated and matured by incubating these with a specific type of T cell: the γδ T cells.

γδ T cells are rapidly activated by bacterial products and subsequently release cytokines such as TNF-α and interferon (IFN)-γ (6-9). Indeed, unlike classical αβT cells, γδ T cells have the ability to interact with non-processed antigens (10). For human γδT cells expressing Vγ9 and Vδ2-encoded receptors, major ligands are represented by phosphoantigens which stimulate their proliferation and their secretion of cytokines (11-15). Bromohydrin pyrophosphate (BrHpp) is a synthetic phosphoantigen which was recently shown to efficiently induce activation of human Vγ9/Vδ2 T cells (16).

The inventors found that especially these γδ T cells have a surprising effect on the activation of monocyte-derived DC. In addition, the inventors showed that the induction of said cells using BrHpp further increased this stimulating effect. In this patent application, the inventors tried to unravel which molecules are mediating the γδT cell-DC interactions.

The present invention identifies a new strategy to improve the ability of DC to elicit T cell responses.

These aims have been met by following embodiments.

### DETAILED DESCRIPTION OF THE INVENTION

The present invention relates to a method for the differentiation and/or maturation of immature myeloid dendritic cells (DC) into HLA-DR, CD86, C083 and IL12 (p40) dendritic cells comprising incubating said DC with δγ T cells. With ‘immature myeloid dendritic cells’ it is meant cells which are cells of the myeloid lineage involved in antigen presentation including monocytes, other dendritic cell precursors and dendritic cells themselves. The effect of δγ T cells on DC was only studied for myeloid cells. Nevertheless, the inventors do not exclude the possiblility that the method for the differentiation and/or maturation of immature myeloid DC, as suggested in present patent application, can be applied for the differentiation and/or maturation of immature lymphoid DC. This has to be interpreted throughout the whole patent application.

There are two main sources of DC precursors: CD34+ stem cells and peripheral blood (PB) monocytes. The main constraints of generating DC from stem cells is that the culture time is long and obtaining CD34+ cells requires mobilization of the patient. Therefore a preferred embodiment of the present invention is to use monocytes as a DC precursor. Respective DC precursor can be induced (see below) creating immature myeloid dendritic cells. Nevertheless, these immature myeloid dendritic cells are not fully mature and probably induce the immunological response only partially when an antigen is present. To characterize additional factors, the inventors investigated the effect of specific immune cells on the differentiation and/or maturation of said cells.

The inventors found surprisingly that coculture of DC with γδ T cells resulted in the upregulation of HLA-DR, CD86, CD83 surface markers, indicating that DC undergo some degree of maturation under the influence of γδT cells.

In addition, freshly isolated γδT cells induced the production of IL-12 (p40) but did not elicit IL-12 (p70) production. Therefore, the inventors defined the differentiated cells of present invention, using δγ T cells, as HLA-DR, CD86, CD83 and IL12 (p40) dendritic cells.

Until now, δγ T cells are isolated from fresh blood. Nevertheless, for the method as described by the invention, also in vitro induced γδ T cells, or cells which can be differentiated into γδ T cells, can be used.

The inventors also points towards the fact that the method according to present invention for the differentiation and/or maturation of immature myeloid dendritic cells (DC) into HLA-DR, CD86, CD83 and IL12 (p40) dendritic cells can be performed in vitro or in vivo. For example, γδ T cells can be injected in patients resulting in the in vivo production of HLA-DR, CD86, CD83 and IL12 (p40) dendritic cells. The inventors suggest that these HLA-DR, CD86, CD83 and IL12 (p40) dendritic cells have a higher capacity to induce efficient Th 1-type and CTL responses compared to already known dendritic cells.

Preferentially, said immature myeloid DC are derived from monocytes through cytokine treatment chosen from IL-4/GM-CSF or IFN-β/IL-3 or functional analogues thereof. It has been previously shown that monocytes can be differentiated in myeloid dendritic cells using a combination of specific cytokines: IL-4 and GM-CSF, or, IFN-β and IL-3 creating IL-4/GM-CSF DC, L-3/IFN-β DC, respectively. IL-3 and IFN-β DC express markers of the myeloid lineage (CD11c, CD14, and CD33) and induce high levels of HLA class I and class II molecules, CD40, CD54, CD 80 and CD86, and IL-3Rα (CD123). Contrary to IL-4/GM-CSF DC, IL-β/IFN-β DCs show much higher levels of IL-3Rα. Conversely, CD1a is expressed on IL-4/GM-CSF DC but not on IL-3/IFN-β DC.

According to the invention, said cytokines are added simultaneously, sequentially or separately with the δγ T cells to the monocytes. In the examples, evidence shown that said immature myeloid DCs are further maturated by the γδ T cells interaction. Nevertheless, the inventors do not exclude the possibility that a similar result may be obtained by first contacting the monocytes with the γδ T cells prior to the cytokine treatment. In addition, it is obvious for a person skilled in the art that both steps may be performed simultaneously.

It is clear from present invention that specific cytokines, secreted by the γδ T cells, make the DC differentiate. The inventors showed in their examples that TNFα and IFN-γ are responsible for observed effects, Nevertheless, the inventors have evidence that there are more factors including interactions between membrane-bound molecules provided by these γδ T cells which makes DC to differentiate further.

In the examples of present invention, a cell ratio of 1:1 for immature myeloid DC: γδ T-cells is used. It is obvious for a person skilled in the art that, variation in this cell ratio is possible. One needs to take in account that, if only a small number of cells are provided, the maturation of these DC can not be performed anymore. This may be explained by the fact that a minimal concentration of these secreted and/or cell factors are necessary to induce further differentiation and/or maturation of DC. Therefore the inventors suggest in the present invention to use a cell ratio of the δγ T cells over the monocytes/dendritic cells between 0.1 and 10. For example, the cell ratio of the δ T cells over the monocytes/dendritic cells is 1:1.

The present patent application also describes a method for the differentiation and/or maturation of immature myeloid dendritic cells (DC) into HLA-DR, CD86, CD83 and IL12 (p70) dendritic cells comprising incubating said DC with activated δγ T cells.

The inventors found that coculture of DC with activated γδ T cells resulted in the upregulation of HLA-DR, CD86, CD83 surface markers. This upregulation is comparable with the increase of said surface markers on DC when using non-activated γδ T cells.

As mentioned above non-activated γδ T cells could stimulate IL12(p40) synthesis in DC. Present invention illustrates that superinduction of IL-12 (p40) synthesis in DC is possible when γδ T cells and DC are cocultured in presence of a γδ T-cell activator (for instance BrHpp). Surprisingly, the inventors found that γδT cells, stimulated with BrHpp, did elicit IL-12 (p70) production by DC; this in contrast to the induction of only IL12(p40) when treated with non-preactivated γδ T cells. Therefore, the inventors defined the differentiated cells of present invention, using activated γδ T cells, as HLA-DR, CD86, CD83 and IL12 (p70) dendritic cells. Also in this method γδ T cells from fresh blood, in vitro induced γδ T cells, or cells which can be differentiated into γδ T cells can be used.

The inventors point towards the fact that the method according to present invention for the differentiation and/or maturation of immature myeloid dendritic cells (DC) into HLA-DR, CD86, CD83 and IL12 (p70) dendritic cells comprising incubating said DC with activated γδ T cells can be performed in vitro or in vivo. For example, activated γδ T cells or γδ T cells supplemented with an activating agent, can be injected in patients resulting in the in vivo production of HLA-DR, CD86, CD83 and IL12 (p70) dendritic cells. Alternatively, the activating agent can be injected ‘as such’ resulting in the activation of endogenous γδ T cells. The inventors suggest that these HLA-DR, CD86, CD83 and IL12 (p70) dendritic cells have an even higher capacity, compared to the HLA-DR, CD86, CD83 and IL12 (p40) dendritic cells, to induce efficient Th1-type and CTL responses.

According to present invention, said activated γδ T cells are produced by treating γδ T cells with an activating agent chosen from microbial products or derivatives thereof. Microbial products such as phosphoantigens are produced by Gram-positive and Gram-negative bacteria and also by some eukaryotic parasites and plants. Mycobacterium tuberculosis produces four distinct phosphoantigens. These molecules share a moiety that is responsible for the potent stimulation of γδ-T cells seen in tuberculosis patients. The structure of this common core is 3-formyl-1-butyl-pyrophosphate, a recently described phosphoester. Synthetic analogues of natural phosphoantigens are also known. Recently Espinosa et al. have developed a synthetic reagent called BrHpp whose biological properties on human γδ T cells are optimized compared to those of 3-formyl-1-butyl-pyrophosphate. As the inventors in present patent application showed that δγ-T cells communicate with DC, BrHpp was used to study the effect of BrHpp-treated γδ-cells. The inventors found that this synthetic analogue could also further stimulate the DC maturation. Therefore, said derivate, bromohydrin pyrophosphate (BrHpp), is used as an example in present invention to further induce DC maturation. In addition, it is likely that γδ T cells have to be activated to induce DC maturation in vivo. Therefore, the inventors propose BrHPP as an adjuvant able to boost Th1 and CTL responses through its ability to induce DC maturation.

According to present invention, BrHpp may be present in the method of present invention at a concentration between 10 and 1000 nM. When BrHpp is present in a too low concentration, no induction is expected. For example, BrHpp may be present at a concentration of 200 nM.

Also here, immature myeloid DC, derived from monocytes through cytokine treatment chosen from IL-4/GM-CSF or IFN-β/IL-3, or functional analogues thereof, may be used to prepare HLA-DR, CD86, CD83 and IL12 (p70) dendritic cells.

According to present invention, said cytokines may be added simultaneously, sequentially or separately with the δγ T cells to the monocytes. In the examples, evidence is shown that said immature myeloid DCs are further maturated by the activated γδ cells interaction. Nevertheless, the inventors do not exclude the possibility that a similar result may be obtained by first contacting the monocytes with the γδ T cells prior to the cytokine treatment. In addition, it is obvious for a person skilled in the art that both steps may be performed simultaneously or that the activating agent, γδ T cells and monocytes are mixed at the same time.

According to the present invention, the cell ratio of the activated δγ T cells over the monocytes/dendritic cells is between 0.1 and 10. For example, the cell ratio of the activated δγ T cells over the monocytes/dendritic cells may be 1:1.

The present invention further contemplates a population of HLA-DR, CD86, CD83, IL12 (p40) dendritic cells and/or a population of HLA-DR, CD86, CD83, IL12 (p70) dendritic cells obtainable by a method according to present invention.

In present invention experimental evidence is given showing that non-activated γδT cells induce the production of IL-12 (p40) in immature myeloid DC. In contrast, activated γδT cells induced the production of IL-12 (p70) by DC, an effect that involved IFN-γ production stimulated by BrHpp. Therefore, the present invention also relates to a method to produce IL12 (p40) or IL12 (p70) using a population of HLA-DR, CD86, CD83 and IL12 (p40) dendritic cells or HLA-DR, CD86, CD83 and IL12 (p70) dendritic cells, respectively. This production can be performed in vitro or in vivo.

The relevance of this finding to DC function is further demonstrated in present invention by the increased production of IL-5 or IFN-γ by allogenic T cells (CD4⁺ T cells) when stimulated in mixed leucocyte reaction LR with DC pre-incubated with nonactivated or activated γδT cells, respectively. Therefore the present invention also provides a method to produce (further induce) IL-5 by alloreactive T cells using a population of HLA-DR, CD86, CD83 and IL12 (p40) dendritic cells according to present invention. The present invention also provides a method to produce (further induce) IFN-γ by alloreactive T cells using a population of HLA-DR, CD86, CD83 and IL12 (p70) dendritic cells according to present invention. This production can be performed in vitro or in vivo. With ‘alloreactive T cells’ is meant T cells specifically recognizing foreign major histocompatibility molecules.

The present invention describes a method for obtaining a population of HLA-DR, CD86, CD83 and IL12(p40) dendritic cells, comprising at least the following steps:
(a) isolating monocytes from a patient,
(b) incubating said monocytes in the presence of IFN-D/IL-3, GM-CSF/IL-4 or functional analogues thereof, producing a population of immature myeloid dendritic cells,
(c) isolating γδ T cells, and,
(d) contacting immature myeloid dendritic cells of step (b) with T cells of step (c), whereby said contact can be performed directly or indirectly. (claim 21)

There are two main sources of DC precursors: CD34+ stem cells and peripheral blood (PB) monocytes. The main constraints of generating DC from stem cells is that the culture time is long and obtaining CD34+ cells requires mobilization of the patient. Therefore a preferred embodiment of the present invention is to use monocytes as a DC precursor. These cells can normally when present in blood differentiate into DC after a 7 day culture in presence of GM-CSF/IL4 or In presence of IL3/IFN-β. Different techniques might be used to isolate monocytes from the blood as known by the person skilled in the art. With the term “population” is meant HLA-DR, CD86, CD83 and IL12(p40) dendritic cells as such, a group of HLA-DR, CD86, CD83 and IL12(p40) dendritic cells which may be different in other characteristics, or a group of cells comprising HLA-DR, CD86, CD83 and IL12(p40) dendritic cells. Also one cell is not excluded from this definition. It is important to mention that, the inventors do not exclude the fact that monocytes can be further differentiated and maturated in vivo by injecting the immature myeloid dendritic cells and γδ T cells as such into the patient.

The inventors demonstrated that cell to cell contact is not required for the induction of DC maturation by γδT cells as it was also observed when the two cell populations were seeded in transwells. Nevertheless, cell to cell contacts involving membrane-bound molecules could also participate as the residual production of IL-12 in the presence of anti-IFN-γ Ab decreased when cells were separated in transwells (data not shown). According to present invention, the contacting of step may be performed for 24 hours.

However it is evident for a person skilled in the art that variation on this incubation time is possible. Present inventors suggest to perform the incubation preferentially between 8 and 48 hours.

The present invention provides a method for obtaining a population of HLA-DR, CD86, CD83 and IL12(p70) dendritic cells, comprising at least the following steps:
(a) isolating monocytes from a patient,
(b) incubating said monocytes in the presence of IFN-β/IL-3, GM-CSF/IL-4 or functional analogues thereof, producing a population of immature myeloid dendritic cells,
(c) isolating γδ T cells,
(d) culturing T cells of step (c) with microbial products or derivatives thereof as defined above, thereby activating said γδ T cells, and,
(e) contacting dendritic cells of step (b) with T cells of step (d), whereby said contact is performed directly or indirectly.

According to present invention, the contacting of step may be performed for 24 hours.

However it is evident for a person skilled in the art that variation on this incubation time is possible. Present inventors suggest to perform the incubation preferentially between 8 and 48 hours.

According to present invention HLA-DR, CD86, CD83 and IL12(p40) dendritic cells may further be treated to produce antigen presenting dendritic cells. Consequently the method as described by the present invention comprises at least the following steps:
(a) isolating monocytes from a patient,
(b) incubating said monocytes in the presence of IFN-β/IL-3, GM-CSF/IL-4 or functional analogues thereof, producing a population of immature myeloid dendritic cells,
(c) isolating γδ T cells,
(d) contacting dendritic cells of step (b) with T cells of step (c), whereby the contact is performed directly or indirectly, and,
(e) presenting a peptide on the surface of said dendritic cells.

According to present invention HLA-DR, CD86, CD83 and IL12(p70) dendritic cells may further be treated to produce antigen presenting dendritic cells. Consequently the method as described by the present invention comprises at least the following steps:
(a) isolating monocytes from a patient,
(b) incubating said monocytes in the presence of IFN-β/IL-3, GM-CSF/IL-4 or functional analogues thereof, producing a population of immature myeloid dendritic cells,
(c) isolating of γδ T cells,
(d) culturing T cells of step (c) with microbial products or derivatives thereof, thereby activating said γδ T cells,
(e) contacting dendritic cells of step (b) with T cells of step (d), whereby said contact is performed directly or indirectly, and,
(f) presenting a peptide on the surface of said dendritic cells.

Depending on the specific treatment as described below, antigens are specific molecules present on cells selected from the group consisting of a cancer cell, a bacteria, a parasitically infected cell and a virally infected cell. These antigens can be large molecules which are processed by the DC to load MHC molecules, or can be smaller molecules (i.e. peptides) which are immediately loaded onto the MHC molecules. Several approaches have been used to arm DC with target antigen for use in clinical trials. Methods used to approach this step of antigen loading are reviewed by Fong and Engleman ³⁶. Inventors also point out that, HLA-DR, CD86, CD83 and IL12(p70) dendritic cells can be produced in vivo by injecting γδ T cells in combination with an antigen into the patient.

The capacity of presenting a peptide on the surface of said dendritic cells according to present invention can for example be achieved by contacting said dendritic cell with at least part of an antigen differentially expressed on a cell. This cell can be a cell selected from the group consisting of a cancer cell, a bacterial cell, a parasitically infected cell and a virally infected cell. Antigens are delivered from these to the DC resulting in the activation of the DCs.

Alternatively, the capacity of presenting a peptide on the surface of said dendritic cells can be achieved by pulsing said dendritic cells with antigenic proteins, by loading said dendritic cells with antigenic peptides or can be achieved by transforming/transducing said dendritic cells by nucleic acid molecules coding for at least part of said antigen. With “pulsing” is meant that DC are activated by these antigens and enter into the MHC class II and/or MHC class I processing pathway. Transformation of DC can be achieved using electric pulses, liposomes or other techniques as known by the person skilled in the art. Viral vectors allow the transduction of cells. With viral vectors also retroviral, adenoviral and adeno-associated vectors are meant.

Transformation/transduction of the cells allows introduction of DNA encoding the antigen and when appropriate expression signals are present said antigen is made in the cell and brought through the endogenous mechanisms to the surface of the transformed/transduced dendritic cell. As a result of this an antigen-presenting HLA-DR, CD86, CD83 and IL12(p40) dendritic cell or an HLA-DR, CD86, CD83 and IL12(p70) dendritic cells is made.

Alternatively, the capacity of presenting a peptide on the surface of said dendritic cells is achieved by fusing said dendritic cell with cells carrying specific antigens. The production of dendritic-like cell/tumor cell hybrids and hybridomas for inducing anti-tumor response have been described in WO96/30030. This document provides dendritic-like cell/tumor cell hybridomas and pluralities of dendritic-like cell/tumor cell hybrids that confer tumor resistance in vivo. The hybrids and hybridomas are generated by the fusion of tumor cells with dendritic-like cells. For instance, immortal tumor cells from an autologous tumor cell line can be fused with autologous or HLA-matched allogeneic dendritic-like cells. Autologous tumor cell lines can be derived from primary tumors and from their metastases. Alternatively, immortal dendritic-like cells from an autologous or allogeneic HLA-matched dendritic-like cell line can be fused with autologous tumor cells. Autologous dendritic-like cell lines can be prepared from various sources such as peripheral blood and bone marrow. Dendritic-like cell/tumor cell hybridomas and pluralities of hybrids can be directly infused for active immunization of cancer patients against their residual tumor cells. The hybridomas and hybrids can also be used for the in vitro activation of autologous immune cells before their reinfusion into the patient for passive immunization against the tumor cells.

The present invention also proposes a method to provide an activated population of T cells using antigen-presenting HLA-DR, CD86, CD83 and IL12 (p40) dendritic cells obtainable by a method as described above comprising at least the following steps:
(a) isolating monocytes from a patient,
(b) incubating said monocytes in the presence of IFN-β/IL-3, GM-CSF/IL-4 or functional analogues thereof, producing a population of immature myeloid dendritic cells,
(c) isolating γδ T cells,
(d) contacting dendritic cells of step (b) with T cells of step (c), whereby said contact is performed directly or indirectly,
(e) presenting a peptide on the surface of said dendritic cells, thereby providing a population of antigen presenting dendritic cells; and,
(f) activating a population of T cells with said population of antigen presenting dendritic cells of step (e).

The inventors point towards the fact that the activated population of T cells may represent both CD4Th1 and/or CD8 cytotoxic T cells (CTL).

A method for activating a T cell using HLA-DR, CD86, CD83 and IL12 (p70) dendritic cells obtainable by a method as described in the previous claims comprising at least the following steps:
(a) isolating monocytes from a patient,
(b) incubating said monocytes in the presence of IFN-β/IL-3, GM-CSF/IL-4 or functional analogues thereof, producing a population of immature myeloid dendritic cells,
(c) isolating γδ T cells,
(d) culturing T cells of step (c) with microbial products or derivatives thereof, thereby activating said γδ T cells,
(e) contacting dendritic cells of step (b) with T cells of step (d), whereby said contact is performed directly or indirectly,
(f) presenting a peptide on the surface of said dendritic cells, thereby providing a population of antigen presenting dendritic cells; and,
(g) activating a population of T cells with said population of antigen presenting dendritic cells of step (f).

An activated T cell being a T cell (CD3+ cell) proliferating and/or secreting cytokines (IL-2, IL-4, IL-5, IFN-γ, etc.) and/or expressing activation markers (CD25, CD69, HLA-DR, CD40L, etc.). If necessary, an activated T cell can always be separated from the antigen presenting dendritic cell by cell sorting.

In preferred methods according to present invention said T cell is a T helper cell.

The invention also refers to a method as described above wherein the steps of producing a population of cells as described above such as HLA-DR, CD86, CD83 and IL12 (p40) dendritic cells, HLA-DR, CD86, CD83 and IL12(p70) dendritic cells, antigen presenting HLA-DR, CD86, CD83 and IL12(p40) dendritic cells, antigen presenting HLA-DR, CD86, CD83 and IL12(p70) dendritic cells and/or activated T cells using antigen presenting HLA-DR, CD86, CD83 and IL12(p40) dendritic cells or antigen presenting HLA-DR, CD86, CD83 and IL12(p70) dendritic cells are carried out in vitro and/or in vivo.

The present invention also provides a population of antigen-presenting HLA-DR, CD86, CD83 and IL12(p40) dendritic cells, a population of antigen-presenting HLA-DR, CD86, CD83 and IL12(p70) dendritic cells or a population of activated T cell obtainable by a method as described above.

The present invention also relates to a composition for use as a medicament or a cell based product intended for clinical use comprising at least one of the following components according to present invention:
a population of HLA-DR, CD86, CD83 and IL12(p40) dendritic cells,
a population of HLA-DR, CD86, CD83 and IL12(p70) dendritic cells,
a population of antigen-presenting HLA-DR, CD86, CD83, IL 12(p40) dendritic cells,
a population of antigen-presenting HLA-DR, CD86, CD83, IL12(p70) dendritic cells,
a population of antigen-presenting HLA-DR, CD86, CD83, IL12(p40) dendritic cells mixed with antigen,
a population of antigen-presenting HLA-DR, CD86, CD83, IL12(p70) dendritic cells mixed with antigen,
a population of activated T cells obtainable using antigen-presenting HLA-DR, CD86, CD83, IL12(p40) dendritic cells, or,
a population of activated T cells obtainable using antigen-presenting HLA-DR, CD86, CD83, IL12(p70) dendritic cells.

Cell based products are not yet considered as medicament and could be considered as transfusion products in the future.

Therefore the present inventors concluded that upon using this specific method a new type of DC can be formed which is more mature than previously described myeloid DC and which have a superior character in inducing T cell expression. Increased cytokine expression results in a more rapid and efficient stimulation of the immune system, and therefore will be more efficient in eliminating foreign infectious material in a patient.

As IL-12 (p70) is the bioactive form of IL12 (p40) the Inventors assume that HLA-DR, CD86, CD83 and IL12(p70) dendritic cells will have a more important effect on the induction of the Th1 and CTL response compared to the HLA-DR, CD86, CD83 and IL12(p70) dendritic cells.

Monocyte-derived DC primed with tumor antigens are now clinically used in several protocols to induce specific antitumor immunity. Both Th1 and Th2 effector mechanisms have been shown to collaborate with each other in directing an effective antitumor activity. Because of their ability to induce both Th1 and Th2 type responses, the inventors suggest that HLA-DR, CD86, CD83 and IL12 (p40) dendritic cells and HLA-DR, CD86, CD83 and IL12 (p70) dendritic cells might be appropriate to induce efficient tumor immunity.

A composition according to the invention may be supplemented with at least one additional cytokine. According to the present invention, said cytokine is may for instance be chosen from a group comprising IFN-α, IFN-β, IL-3 and IL-12. IL-12 and IFN-α are pivotal cytokines for Th1 differentiation and generation of cytotoxic T cells endowed with potent anti-tumor effects.

The invention implies the use of a compound according to present invention for the preparation of a medicament. Specifically, these compounds can be used for the preparation of a medicament for the treatment and/or prevention of a disease whereby stimulation of the Th1 and/or CTL response is needed comprising a composition as described above. These diseases can be chosen from the group comprising cancer, infections and autoimmune diseases. Investigations showed that the immunologic and clinical effects of antigen-loaded dendritic cells administered as a therapeutic vaccine to patients with cancer. Although DC-based vaccination methods are cumbersome, promising results from clinical trials in patients with malignant lymphoma, melanoma, and prostate cancer suggest that immunotherapeutic strategies that take advantage of the antigen-presenting properties of dendritic cells may ultimately prove both efficacious and widely applicable to human tumors. Also the role of DC in initiating or priming immune responses to viral and bacterial antigens in vivo is well established. It has been demonstrated that human DC, but not monocytes or B cells, can sensitise naïve T cells to soluble protein antigens, enabling the generation of antigen-specific CD4+ helper and CD8+ CTL lines in vitro. CD8+ cytotoxic T lymphocytes (CTL) have been demonstrated to recognize and kill cancer cells in various tumor models. The ability of DC to prime T cells capable of recognizing and killing tumor cells in an antigen-specific fashion has been demonstrated in various animal models. Moreover, DC-based immunization can lead to immunologic memory with protection against subsequent tumor challenges. Fong et al (1997) illustrated that immunizing with self proteins could protect animals against autoimmune reactions.

The present invention also relates to the use of microbial products (such as phosphoester, BrHpp, derivatives or combinations thereof) for the preparation of a medicament for the treatment and/or prevention of a disease whereby stimulation of the Th1 and/or CTL response is needed. Also here, said disease can be chosen from the group consisting of cancer, infections and autoimmune diseases. The inventors point hereby to the fact that BrHpp can be used as an adjuvant to elicit DC maturation in vivo.

The present invention also relates to the pharmaceutical composition comprising at least one of the components according to the invention and optionally a pharmaceutical acceptable carrier, diluent or excipient. Pharmaceutically acceptable carriers include any carrier that does not itself induce the production of antibodies harmful to the individual receiving the composition. Suitable carriers are typically large, slowly metabolizing macromolecules such as proteins, polysaccharides, polylactic acids, polyglycolic acids, polymeric amino acids, amino acid copolymers; and inactive virus particles. Such carriers are well known to those of ordinary skill in the art. A “vaccine” is an immunogenic composition capable of eliciting protection against infections, whether partial or complete. A vaccine may also be useful for treatment of an individual, in which case it is called a therapeutic vaccine. Said vaccine compositions may include prophylactic as well as therapeutic vaccine compositions. The term “therapeutic” refers to be capacity of eliminating or preventing invasive cells.

The present invention also relates to a method of killing a target cell comprising contacting said target cell with a composition. This killing can be performed in vitro or in vivo. Said target cell may for instance be selected from the group consisting of a cancer cell, a bacterial cell, a parasitically infected cell or a virally-infected cell.

The present invention also provides an in vitro screening method using a population of HLA-DR, CD86, CD83 and IL12(p40) dendritic cells, a population of HLA-DR, CD86, CD83 and IL12(p70) dendritic cells, a population of antigen-presenting HLA-DR, CD86, CD83 and IL12(p40) dendritic cells, a population of antigen-presenting HLA-DR, CD86, CD83 and IL12(p70) dendritic cells or a population of activated T cells obtainable by a method as described above. By their potent immunostimulatory properties, DC loaded with tumor or bacterial Ag could be used to activate T cells against unknown poorly immunigenic Ag and thus help to discover them.

According to present invention, a population of HLA-DR, CD86, CD83 and IL12(p40) dendritic cells, a population of HLA-DR, CD86, CD83 and IL12(p70) dendritic cells, a population of antigen-presenting HLA-DR, CD86, CD83 and IL12(p40) dendritic cells, a population of antigen-presenting HLA-DR, CD86, CD83 and IL12(p70) dendritic cells or a population of activated T cell obtainable by a method according to the invention can be used for the preparation of in vitro screening tests.

According to present invention, a method for detecting T cell mediated activity of a target antigenic peptide, comprising at least the following steps:
(a) providing a population of antigen-presenting HLA-DR, CD86, CD83 and IL12(p40) dendritic cells or a population of antigen-presenting HLA-DR, CD86, CD83 and IL12(p70) dendritic cells obtainable according to a method as described above,
(b) contacting a T cell with said dendritic cell, thereby providing an activated T cell,
(c) contacting a target cell with said activated T cell, and,
(d) monitoring the effect of said activated T cell on said target cell, thereby detecting anti-target activity.

The present invention also describes a kit for detecting T cell mediated activity of a target antigenic peptide, comprising at least one of the following components according to present invention:
a population of HLA-DR, CD86, CD83 and IL12(p40) dendritic cells,
a population of HLA-DR, CD86, CD83 and IL12(p70) dendritic cells,
a population of antigen-presenting HLA-DR, CD86, CD83, IL12(p40) dendritic cells,
a population of antigen-presenting HLA-DR, CD86, CD83, IL12(p70) dendritic cells,
a population of antigen-presenting HLA-DR, CD86, CD83, IL12(p40) dendritic cells mixed with antigen,
a population of antigen-presenting HLA-DR, CD86, CD83, IL12(p70) dendritic cells mixed with antigen,
a population of activated T cells obtainable using antigen-presenting HLA-DR, CD86, CD83, IL12(p40) dendritic cells, or,
a population of activated T cells obtainable using antigen-presenting HLA-DR, CD86, CD83, IL12(p70) dendritic cells.

It has been shown that freezing population of said cells did not alter the functional properties of these cells. Also other methods for storage as known by the skilled person in the art can be applied to preserve these cells.

All methods, uses and kits described in present Invention for the detection of T cell mediated activity also relate to the use of a population of HLA-DR, CD86, CD83 and IL12 (p40) dendritic cells and/or HLA-DR, CD86, CD83 and IL12 (p70) dendritic cells as reagent for the purpose of following the immune response in patients who got either DC-vaccines or other vaccines. T cells might be isolated from patients and tested using antigen-presenting HLA-DR, CD86, CD83 and IL12 (p40) dendritic cells or antigen-presenting HLA-DR, CD86, CD83 and IL12 (p70) dendritic cells to analyse if the immunologic response in the patient has been activated. For this purpose PBMC (periferal blood mononuclear cells) or purified T cells might be used. This test system allows the evaluation of any therapy against infections, cancer or auto-immune diseases.

The present invention suggests the use of a composition according to the invention as a vaccine adjuvant and the vaccine adjuvant as such comprising a composition according to the invention.

As pointed out before, compositions according to present invention and/or microbial products, such as BrHpp, can be used as a vaccine adjuvant for the stimulation of DC maturation in vivo.

According to present invention a vaccine comprising the composition as described by the invention can be used to immunize humans or animals against different diseases (adjuvant). Vaccination of patients has already been illustrated and found to be efficacious using peptide-pulsed IL4/GM-CSF DC in cancer patients (Toungouz et al. 1999).

In particular, the present invention describes a method for immunizing humans or animals against a disease comprising administering a vaccine comprising an adjuvant as described above.

In terms of vaccination against infectious diseases, BrHPP might especially be of interest in the newborn in which DC are deficient. The inventors propose that BrHPP might allow to enhance the function of neonatal DC and therefore when incorporated in vaccine formulations might allow in combination with antigens to induce efficient immunity against pathogens (i.e. malaria, tuberculosis, HIV) Therefore, the present invention also relates to a method of immunizing newborns.

The present invention also relates to a method of treatment of cancer, infections and autoimmune diseases comprising the use of at least one of the following compositions according to the invention.

In the design and conduct of above described applications, important considerations include methods for introducing the antigen into MHC class 1 and 11 processing pathways, methods for isolating and activating dendritic cells, route of administration and antigen selection. Because the cell therapy as presented in the present invention needs a specific recognition of the target cell, it is important that indeed the choice of antigen is well considered. Therefore the present invention suggests that the antigen is a tumor specific antigen, an infectious specific antigen or a self-protein when applied in the treatment of cancer, infections (viral, bacterial, parasitical) or autoimmune diseases. In addition, it is important that the compositions are administered to a person in need of treatment in a therapeutically effective amount. Example of antigens that might be considered as tumor antigens are described by Fong and Engleman 2000.

According to the present invention said viral disease is selected from the group of HIV, human Papilloma virus, Ebstein Barr virus and Cytomegalovirus.

According to the present invention said autoimmune disease is selected from the group consisting of multiple sclerosis myasthenia gravis, juvenile chronic arthritis, chronic arthritis, LED, atopic dermatitis and juvenile diabetes. Inventors suggest that probably all autoimmune diseases may be treated or prevented by a method as described by the invention.

According to the present invention, said compositions can be injected into patients using different ways. Injection may for instance be carried out intravenously, intra-lymphoidal or intratumoral, nevertheless, other routes can be used such as subcutaneous injections. It is interesting to mention that in addition to expressing the requisite MHC and costimulatory molecules to prime T cells, the DC cells express appropriate adhesion molecules and chemokine receptors to attract the DC to secondary lymphoid organs for priming. In this respect, inefficient priming could be circumvented by injecting DC directly to secundary lympoid organs through intralymphatic or intranodal injection. The present study gives evidence that especially in cancer treatment intra-tumoral injections will result in more efficient elimination of the tumor. The inventors showed in EP00870273.0 that monocyte-derived IL-3/IFN-β DC are able to trigger apoptosis in tumor cells which is relevant to their therapeutic use as anti-tumor vaccines. Indeed, recent reports demonstrated that human IL-4/GM-CSF DC can process apoptotic cells and cross-present the derived antigens in a MHC-class I restricted fashion, resulting in the induction of efficient cytotoxic T cell responses. Therefore present inventors suggest that antigen-presenting HLA-DR, CD86, CD83 and IL12 (p40) dendritic cells and/or antigen-presenting HLA-DR, CD86, CD83 and IL12 (p70) dendritic cells which are directly injected into tumors may first induce apoptosis of cancer cells, and finally migrate in the lymph nodes where they induce tumor-specific T-cell responses.

These compositions may, for example, be administered parentally or intravenously. The compositions according to the invention for parenteral administration can be, in particular, sterile solutions, aqueous or non-aqueous, suspensions or emulsions, As a pharmaceutically acceptable solution or vehicle propylene glycol, polyethylene glycol, injectable organic esters, for example ethyl oleate, or cyclodextrins may be employed. These compositions can also comprise wetting, emulsifying and/or dispersing agents.

The sterilisation may be carried out in several ways, for example, using bacteriological filter, by incorporating sterilising agents in the composition or by irradiation. They may also be prepared in the form of sterile solid compositions which may be dissolved at the time of use in sterile water or any other sterile injectable medium.

The present invention can also comprise adjuvants which are well known to a person skilled in the art (vitamin C, antioxidant agents, etc.) capable of being used in synergy with the compounds according to the invention in order to improve and prolong the treatments of cancerous tumors.

The invention also relates to a composition comprising a composition according to present invention and another compound as a combined preparation for simultaneous, separate or sequential use for treating cancer, infections and autoimmune diseases.

The present invention also relates to a method for the preparation of a composition as described by present invention comprising following steps:
(a) isolating monocytes from a patient,
(b) incubating said monocytes in the presence of IFN-β/IL-3, GM-CSF/IL-4 or functional analogues thereof in clinical grade conditions, producing a population of immature myeloid dendritic cells,
(c) isolation of γδ T cells, and,
(d) contacting immature myeloid dendritic cells of step (b) with T cells of step (c), in clinical grade conditions,
(e) presenting an antigen on the surface of said dendritic cells in clinical grade conditions, thereby providing a population of antigen presenting dendritic cells; and,
(f) activating a population of T cells with said population of antigen presenting dendritic cells. As described above each of these steps can be performed in vitro and/or in vivo.

Recently improvements were made for the production of DC in clinical-grade conditions. The present inventors described in Toungouz et al 1999³⁸ that the development of closed systems, avoidance of exogenous proteins and respect of standard operating procedures (SOP) is needed to be able to guarantee predefined specifications of the cellular product. In these documents a good manufacturing practice (GMP)-simplified procedure of IL4/GM-CSF DC generation from leukapheresis products in a closed system, using synthetic culture media devoid of non-human protein is described. In analogy to this method, clinical grade HLA-DR, CD86, CD83 and IL12 (p40) dendritic cells or HLA-DR, CD86, CD83 and IL12 (p70) dendritic cells can be prepared.

Unless other wise defined, all technical and scientific terms used herein have the same meaning as commonly understood by one of ordinary skill in the art to which this invention belongs. Exemplary methods and materials are described below, although methods and materials similar or equivalent to those described herein can be used in the practice or testing of the present invention. All publications and other references mentioned herein are incorporated by reference in their entirety. In case of conflict, the present specification, including definitions, will control. The materials, methods, and examples are illustrative only and not intend to be limiting. Other features and advantages of the invention will be apparent from the following drawings, detailed description, and from the claims.

### BRIEF DESCRIPTION OF THE FIGURES

FIG. 1: γδ T cells induce the upregulation of HLA-DR, CD86 and CD83 expression on monocyte-derived DC. Monocyte-derived DC were either cultured in medium alone, in the presence of BrHpp (100 nM), or in the presence of γδ T cells which were prestimulated or not with BrHpp. DC and γδ T cells were also cocultured in transwells. DC cell surface markers were analyzed after overnight coculture using flow cytometry. One representative experiment out of 6 is shown.

FIG. 2: Role of TNF-α in the upregulation of DC surface molecules induced by γδ T cells. Monocyte-derived DC were cocultured with BrHpp-activated γδ T cells in the presence of neutralizing anti-TNF-α (20 ug/ml), anti-IFN-γ (15 ug/ml) mAb or both. After overnight culture, cell surface markers were assessed by flow cytometry. One representative experiment out of 5 is shown.

FIG. 3: γδ T cells induce IL-12 production by DC. Monocyte-derived DC were cocultured alone or in the presence of BrHpp only or cocultured with unstimulated or BrHpp-activated γδ T in absence or presence of either anti-TNF-α, anti-IFN-γ neutralizing mAbs or both. After 24 h of culture, supernatants were assayed for IL12-p40 and p70 levels by ELISA. Results were expressed as mean±SEM of 6 independent experiments.
* p<0.05 compared to DC cultured in medium alone or containing BrHpp
** p<0.05 compared to DC stimulated with BrHPP-activated γδ T cells in the absence of mAb.

FIG. 4: DC were either cultured alone, or in the presence of unstimulated or BrHpp-stimulated γδ T cells, thereafter irradiated (6000 Rads) and finally added to allogenic CD4⁺ T cells. After 5 days, supernatants were assayed by ELISA for IFN-γ and IL-5 levels. Data are shown as mean±SEM of 5 independent experiments. * p<0.05 as compared with DC that were not precultured with γδ T cells.

Table 1: TNF-α and IFN-γ production by γδ T cells.

γδ T cells (7.5 10⁵ cells/500 μl) were either cultured in medium alone or stimulated with BrHpp (200 nM). After 48 h, culture supernatants were assayed by ELISA for TNF-α and IFN-γ levels. Data are shown as mean±SEM of 13 independent experiments.
¹ p<0.003 as compared to medium alone (without BrHpp)
   Table 2: Phenotypic Changes of DC upon Coculture with γδ T cells.

DC cultures were cultured for 24 alone, in the presence of BrHpp (200 nM) only, or with γδ T cells activated by BrHpp as described in example 1. Neutralizing anti-TNF or anti-IFNγ Ab was added at a concentration of 20 and 15 μg/ml, respectively. The expression of HLA-DR, CD86, and CD83 on DC was measured by flow cytometry and expressed as means+/−SEM of mean fluorescence intensity in five independent experiments on different healty donors.
¹ p<0.05 compared to DC cultured alone or with BrHpp only.
² p<0.05 compared to DC cultured with activated γδ T cells in absence of mAb.

### MODES FOR CARRYING OUT THE INVENTION

### EXAMPLE 1

### Material and Methods

Reagents and Medium. The phosphoantigen bromohydrin pyrophosphate (BrHpp) was kindly provided by Innate Pharma (Marseille, France). Culture medium consisted of RPMI-1640 (Life-Technologies, Paisley, Scotland) supplemented with 50 μM mercaptoethanol, 20 μg/ml gentamycin, 2 mM L-glutamine, 1% nonessential amino acids (Life Technologies) and FBS-10% (Perbio, Aalst, Belgium).

Purification of γδ T cells and DC generation. Peripheral blood mononuclear cells (PBMC) from healthy volunteers were isolated by density centrifugation of heparinized blood on Lymphoprep (Nycomed, Oslo, Norway), washed with HBSS, resuspended in culture medium and allowed to adhere In culture flasks for 2 h at 37° C. Non-adherent cells were removed and adherent monocytes were cultured during 6 days in presence of 500 U/ml granulocyte macrophage colony-stimulating factor (GM-CSF) (Leucomax, Schering-Plough Kenilworth, N.J.) and 800 U/ml of IL-4 (Cellgenix, Freiburg, Germany). The resulting cell preparation routinely contained >90% DC as assessed by morphology and FACS analysis.

For γδ T cell isolation, cells expressing γ-δ receptors on their membrane were positively selected using immunomagnetic depletion (Miltenyi Sanvertech, Belgium). Briefly, non-adherent PBMC containing 2 to 5% of γδ T cells were incubated with biotin-conjugated anti-γδ T cell receptor (TCR) antibodies for 15 min at 4° C., washed three times, and then incubated with immunomagnetic beads coated with streptavidin. Positively selected populations routinely contained more than 90% viable γδ T cells as assessed by flow cytometry. Those cells were positive for CD3 and γδ TCR and expressed neither CD25 not CD40L.

Cell culture conditions. γδ T cells (7.5 10⁵ cells/500 μl) were cultured for 24 h in flat-bottomed 24-well plates in culture medium supplemented or not with BrHpp (200 nM). Autologous DC (10⁶ cells/500 μl) were added to γδ T cell cultures for another 24 h and analyzed for the expression of surface markers and for their ability to release cytokines. In parallel, DC (10⁶ cells/ml) were cultured for 24 h in 24-well plates either in medium alone, or in presence of BrHpp (100 nM). In some experiments, anti-TNF-α (20 μg/ml) or anti-IFN-γ (15 μg/ml) neutralizing monoclonal antibody (mAb) or their isotypic control used at similar concentration (Biosource Fleurus, Belgium) were added to DC-γδ T cell cocultures. In parallel, DC and γδ T cells were cocultured in a transwell culture system (Costar, Belgium).

Determination of cytokine levels. TNF-α, IL-12 p-40 and IFN-γ levels in culture supernatants were determined by ELISA kits from Biosource. IL-12 p70 levels were measured using the Endogen Elisa kit (Endogen, Belgium).

Immunophenotyping by flow cytometry. Monocyte-derived DC were stained using PE-labeled specific mAb HLA-DR, CD86, CD83 (Beckton-Dickinson, Mountain view, CA). FITC-conjugated anti-TCR γδ mAb (Becton-Dickinson, San Jose, Calif.) was used to assess γδ T cell purity and to exclude them in flow cytometry analysis of DC in DC-γδ T cell cocultures. Briefly, 5×10⁵ cells were incubated with the relevant mAbs or their isotype-matched controls for 20 min at 4° C., washed and fluorescence intensity was analyzed using a FACS-Calibur (Becton-Dickinson).

Mixed leucocyte reactions (MLR). 2×.10⁵ CD4⁺ T cells purified from the PBMC of healthy donors using Miltenyi beads were seeded in mixed cultures with irradiated (6000 rads) allogenic DC (2×.10⁴ DC/well). DC were either unstimulated or pre-activated by coculture for 24 h with autologous γδ T cells in presence of 200 nM BrHpp. After 5 days, mixed leucocyte reaction (MLR) supernatants were assayed for IFN-γ and IL-5 by ELISA.

Statistical analysis. Statistical analysis was performed using a non parametric Wilcoxon test.

### EXAMPLE 2

### Human γδ T Cells Induce Upregulation of HLA-DR, CD86 and CD83 Expression on Monocyte-Derived Dendritic Cells: Role of TNF-α

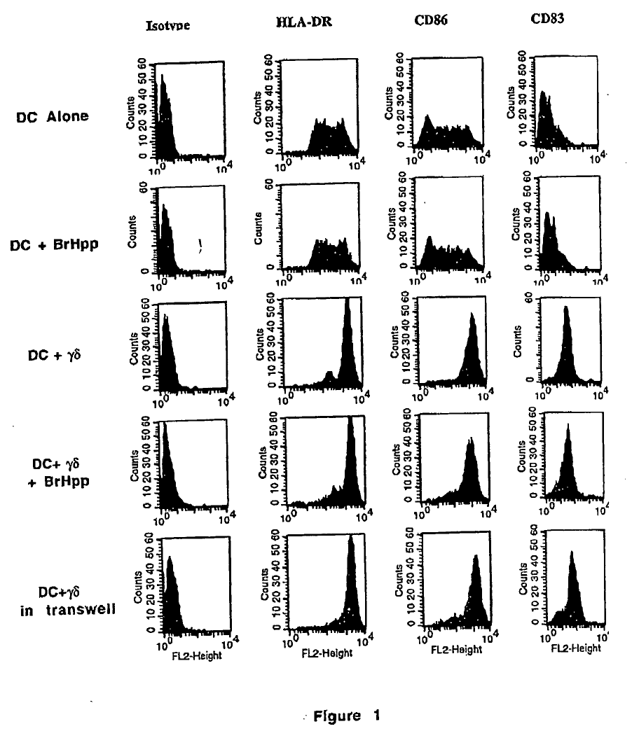

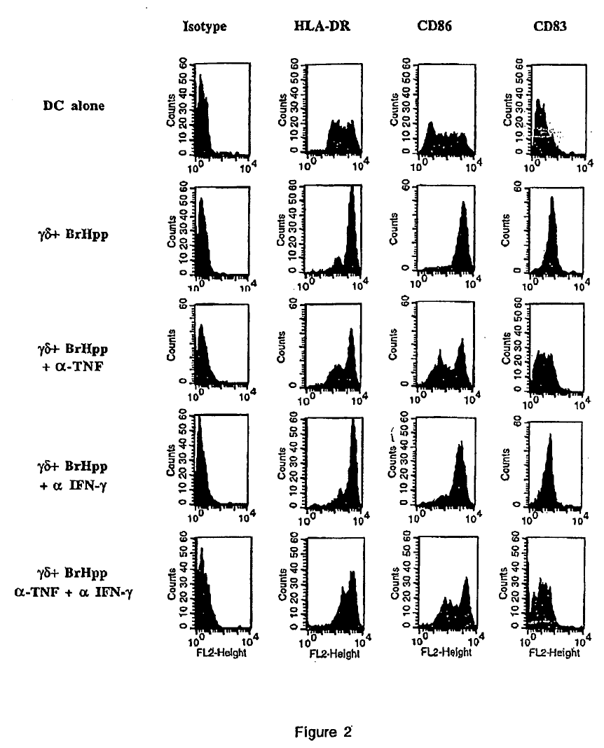

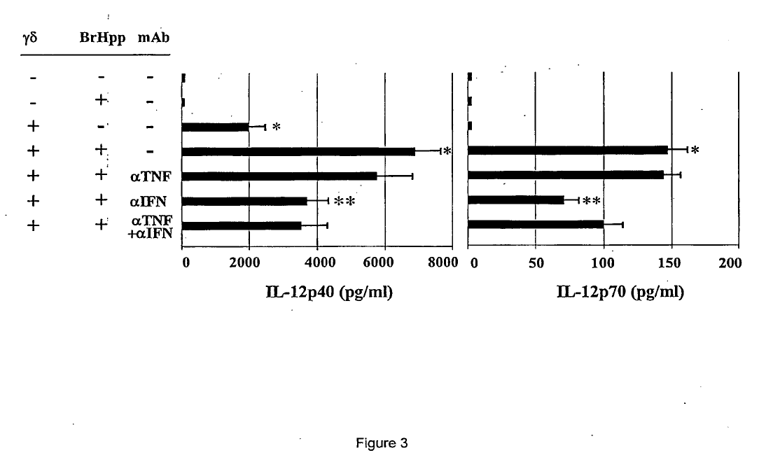

In a first set of experiments, the inventors analyzed by flow cytometry HLA-DR, CD86 and CD83 expression on dendritic cells derived from PBMC cultured in IL-4 and GM-CSF. As shown in FIG. 1 and table 2, coculture of DC with γδ T cells resulted in the upregulation of these surface markers, indicating that DC undergo some degree of maturation under the influence of γδT cells. Preactivation of γδT cells with BrHpp did not result in a further increase of this effect. Cell to cell contact was not required for the induction of DC maturation by γδT cells as it was also observed when the two cell populations were seeded in transwells (FIG. 1 and table 2). As γδT cells are known to secrete TNF-α, the inventors considered the possibility that this cytokine was responsible for the action of γδT cells on DC. Indeed, the inventors found that γδT cells directly isolated from blood produced significant amounts of TNF-α, even in absence of in vitro stimulation (table 1). This In vitro production of TNF-α by purified γδ T cells could be related to the isolation procedure. BrHpp further increased this basal production of TNF-α and also induced IFN-γ secretion by γδ T cells (table 1). Addition of a neutralizing anti-TNF-α mAb to the DC-γδT cells cocultures clearly reduced the upregulation of HLA-DR, CD86 and CD83 whereas anti-IFN-γ mAb failed to do so (FIG. 2 and table 2). These data establish a key role for TNF-α in the maturation of DC elicited by γδ T cells.

### EXAMPLE 3

### γδ T Cells Stimulate IL-12 Production by Dendritic Cells: Involvement of IFN-γ

The capacity of DC to induce efficient Th1-type and CTL responses is linked at least in part to their synthesis of IL-12. The inventors therefore investigated in coculture experiments the impact of γδ T cells on the synthesis by DC of IL-12 (p40) and IL-12 (p70), the bioactive heterodimeric form of the cytokine. Freshly isolated γδT cells induced the production of IL-12 (p40) even in the absence of stimulation by BrHpp. In the presence of BrHpp, a 3-fold increase in IL-12 (p40) levels was observed, and the induction of IL-12 (p70) synthesis was also detected in this setting (FIG. 3). As BrHpp had no effect on DC cultured in absence of γδ T cells (FIG. 3), the inventors concluded that activation of γδ T cells by BrHpp was responsible for the induction of IL-12 synthesis when γδ T cells and DC were cocultured in the presence of BrHpp. Whereas freshly isolated γδ T cells did not elicit IL-12 (p70) production by DC, they did so when stimulated by BrHpp (FIG. 3). The addition of a neutralizing anti-IFN-γ mAb significantly inhibited the induction of both IL-12 (p40) and IL-12 (p70) synthesis, whereas an anti-TNF-α mAb had no effect (FIG. 3). The inventors conclude from these experiments that γδ T cells induce IL-12 production by DC and that this effect partially involves IFN-γ. Cell to cell contacts involving membrane-bound molecules could also participate as the residual production of IL-12 in the presence of anti-IFN-γ Ab decreased when cells were separated in transwells (data not shown). CD40-CD40L interactions were not responsible for the residual production of IL-12 in the presence of anti-IFNγ mAb, as CD40L was not found by flow cytometry at the surface of the δγ T cells even after BrHpp stimulation, and the addition of a blocking anti-CD-40L mAb did not modify IL-12 production in DC-γδ T cell cocultures (data not shown).

### EXAMPLE 4

### Increased Allostimulatory Capacity of DC Cultured in Presence of Activated γδ T Cells

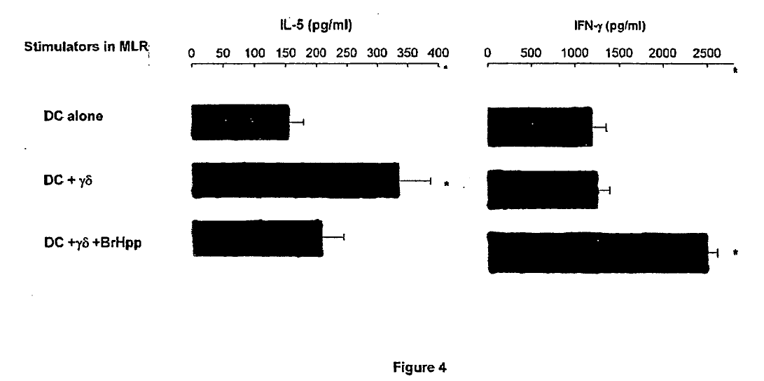

In order to determine the relevance of the effects of γδ T cells on DC, DC pre-cultured in presence of unstimulated or BrHpp-activated γδ T cells were irradiated and then seeded as stimulators in mixed leucocyte reaction (MLR) with allogenic CD4⁺ T cells for 5 days. Compared to control DC, DC pre-cultured with unstimulated γδ T cells induced the production of increased amounts of IL-5 but not IFN-γ by alloreactive T cells (FIG. 4). On the other hand, DC pre-cultured with BrHpp-activated γδT cells induced significantly higher levels of IFN-γ in MLR whereas IL-5 levels were not significantly modified (FIG. 4). These data might be related to the different levels of IL-12 produced by DC pre-cultured in presence of unstimulated or BrHPP-activated γδT cells. Indeed, the low levels of IL-12 produced by the former should favor Th2-type responses whereas the increased levels produced by the latter should promote Th1-type responses (17). In each case, the immunogenic potential of DC increased following the interactions with γδ T cells, probably as a consequence of the upregulation of key surface molecules reported here above.

### EXAMPLE 5

### Discussion

The findings reported by the inventors establish a new link between innate immunity and the induction of acquired T cell responses. Indeed, γδ T cells are rapidly activated in the course of several infections where they provide a primary protection (18, 19). The activation of DC that they simultaneously induce might be critical for the development of efficient CD4⁺ and CD8⁺ T cell responses. Furthermore, the observation made by the inventors that synthetic ligands of the Vγ9/Vδ2 TCR induce DC activation suggest that these agents should be considered as potential vaccine adjuvants. They might be of special interest for early life immunization against intracellular pathogens as the inventors recently demonstrated that neonatal DC display a defect in IL-12 (p70) synthesis which can be corrected by IFN-γ (20). Along the same line, the inventors currently investigate the possibility that the efficient Th1 responses induced in human newborns by the Bacillus Calmette-Guerin (BCG) vaccine (21) are related at least in part to activation of γδ T cells by mycobacterial phosphoantigens.

### EXAMPLE 6

### Summary of the Invention

γδ T cells are known to be involved in the innate immune defenses against infectious micro-organisms. In this patent application, the inventors considered that γδT cells could also influence acquired immunity by interacting with dendritic cells (DC) in the early phase of the immune response. To investigate this hypothesis, γδ T cells isolated from peripheral blood of healthy volunteers were cocultured with autologous monocyte-derived dendritic cells which were subsequently analyzed for their expression of key surface molecules and for their production of IL-12. First, the inventors found that γδT cells induced the upregulation of HLA-DR, CD86 and CD83 on DC. This effect did not require cell to cell contact and could be blocked by a neutralizing anti-TNF antibody. In the same system, γδT cells induced the production of IL-12 (p40) but not IL-12 (p70) by DC. The inventors then assessed the consequence of γδT cell activation by the synthetic phosphoantigen bromohydrin pyrophosphate (BrHpp). γδT cells activated by the synthetic phosphoantigen bromohydrin pyrophosphate (BrHpp) induced the production of IL-12 (p40) and IL-12 (p70) by DC, an effect that involved IFN-γ production. The relevance of this finding to DC function was demonstrated by the increased production of IFN-γ by alloreactive T cells when stimulated in MLR with DC pre-incubated with activated γδT cells. The inventors conclude that γδT cell activation might result in DC maturation and thereby in enhanced αβ T cell responses.

### REFERENCES

1. Rescigno, M., Winzler, C., Della, D., Mutini, C., Lutz, M. and Ricciardi-Castagnoli. P., Dendritic cell maturation is required for initiation of the immune response. J. Leukoc. Biol. 1997. 61: 415-21.
2. Banchereau, J. and Steinman, R. M., Dendritic cells and the control of immunity. Nature 1998. 392:245-251.
3. Rescigno, M., Martino, M., Sutherland, C. L., Gold, M. R. and Ricciardi-Castagnoli, P., Dendritic cell survival and maturation are regulated by different signaling pathways. J. Exp. Med. 1998.188: 2175-80.
4. De Smedt, T., Pajak, B., Muraille, E., Lespagnard, L., Heinen, E., De Baetselier, P., Urbain, J., Leo, O. and Moser, M., Regulation of dendritic cell numbers and maturation by lipopolysaccharide in vivo. J. Exp. Med. 1996.184:1413-1424.
5. Verhasselt, V., Buelens, C., Willems, F., De Groote, D., Haeffner-Cavaillon, N. and Goldman, M., Bacterial lipopolysaccharide stimulates the production of cytokines and the expression of costimulatory molecules by human peripheral blood dendritic cells: evidence for a soluble CD14-dependent pathway. J. Immunol. 1997. 158: 2919-2925.
6. Leclercq, G., and Plum, J., Stimulation of TCR V3 cells by gram-negative bacteria. J. Immunol. 1995. 154: 5313-5319.
7. Nitta, T., Imai, H., Ogasawara, Y. and Nakano, M., Mitogenicity of bacterial lipopolysaccharide on the T lymphocyte population bearing the T cell receptor. J. Endotoxin Res. 1994. 1: 101-107.
8. Skeen, M. J. and Ziegler, H. K., Induction of murine peritoneal/T cells and their role in resistance to bacterial infection. J. Exp. Med. 1993.178: 971-984.
9. Mombaerts, P., Arnoldi, J., Russ, F., Tonegawa, S. and Kaufmann, S. H. E., Different roles of and T cells in immunity against an intracellular bacterial pathogen. Nature 1993. 365: 53-55.
10. Holoshitz, J., Romzek, N. C., Jia, Y., Wagner, L., Vile, L. M., Chen, S. J., Wilson, J. M., Karp, D. R., MHC-independent presentation of mycobacteria to human gamma delta T cells. Int. Immunol. 1993. 5: 1437-1443.
11. Barnes, P. F., Abrams, J. S., Lu, S., Sieling, P. A., Rea, T. H., Modlin, R. L., Patterns of cytokine production by mycobacterium-reactive human T-cell clones. Infect. Immun. 1993. 61: 197-203.
12. Sireci, G., Champagne, E., Fournie, J. J., Dieli, F., Salerno, A., Patterns of phosphoantigen stimulation of human Vgamma9/Vdelta2 T cell clones include Th0 cytokines. Hum. Immunol. 1997.58: 70-82.
13. Lang, F., Peyrat, M. A., Constant, P., Davodeau, F., David-Ameline, J., Poquet, Y., Vie, H., Fournie, J. J., Bonneville, M., Early activation of human V gamma 9V delta 2 T cell broad cytotoxicity and TNF production by nonpeptidic mycobacterial ligands. J. Immunol. 1995.154: 5986-5994.
14. Ferrick, D. A., Schrenzel, M. D., Mulvania, T., Hsieh, B., Ferlin, W. G., Lepper, H., Differential production of interferon-gamma and interleukin-4 in response to Th1- and Th2-stimulating pathogens by gamma delta T cells in vivo. Nature 1995. 373: 255-257.
15. Morita, C. T., Beckman, E. M., Bukowski, J. F., Tanaka, Y., Band, H., Bloom, B. R., Golan, D. E., Brenner, M. B., Direct presentation of nonpeptide prenyl pyrophosphate antigens to human gamma delta T cells. Immunity 1995. 3: 495-507.
16. Espinosa, E., Belmant, C., Pont, F., Luciani, B., Poupot, R., Romagné, F., Brailly, H., Bonneville, M., Fournié, J. J., Chemical synthesis and biological activity of bromohydrin pyrophosphate a potent stimulator of human γδ T cells. J. Biol. Chem. 2001. 276:18337-18344.
17. Kalinski, P., Hilkens, C. M., Wierenga, E. A., Kapsenberg, M. L., T-cell priming by type-1 and type-2 polarized dendritic cells: the concept of a third signal. Immunol. Today 1999. 20: 561-567.
18. Hiromatsu, K., Yoshikai, Y., Matsuzaki, G., Ohga, S., Muramori, K., Matsumoto, K., Bluestone, J. A. and Nomoto. K., A protective role of γδ T cells in primary infection with Listeria monocytogenes in mice. J. Exp. Med. 1992. 175: 49-54.
19. Ohga, S., Yoshikai, Y., Takeda, Y., Hiromatsu, K. and Nomoto, K., Sequential appearance of /- and /-bearing T cells in the peritoneal cavity during an i.p. infection with Listeria monocytogenes. Eur. J. Immunol 1990. 20: 533-538.
22. Goriely, S., Vincart, B., Stordeur, P., Vekemans, J., Willems, F., Goldman, M., and De Wit, D., Deficient IL-12(p35) gene expression by dendritic cells derived from neonatal monocytes. J. Immunol 2001.166:2141-2146.

21. Marchant, A., Goetghebuer, T., Ota, M. O., Wolfe, I., Ceesay, S. J., De Groote, D., Corrah, T., Bennett, S., Wheeler, J., Huygen, K., Aaby, P., McAdam, K. P. and Newport, M. J., Newborns develop a Th1-type immune response to Mycobacterium bovis bacillus Calmette-Guérin vaccination. J. Immunol. 1999. 163: 2249-2255.

TABLE 1
TNF-α and IFN-γ production by γδ T cells

| BrHpp added | TNF-α (pg/ml) | IFN-γ (pg/ml) |
|---|---|---|
| None | 12989 ± 1236 | 98 ± 16 |
| 200 nM | 17406 ± 1290¹ | 452 ± 76¹ |

TABLE 2
Phenotypic Changes of DC upon Coculture with γδ T cells

| DC cocultures | HLA DR | CD86 | CD83 |
|---|---|---|---|
| Alone | 645 +/− 324 | 326 +/− 219 | 80 +/− 17 |
| BrHpp | 610 +/− 354 | 308 +/− 205 | 69 +/− 14 |
| Activated γδ T cells | 1143 +/− 549¹ | 612 +/− 505¹ | 140 +/− 35¹ |
| Activated γδ T cells in | 1358 +/− 417¹ | 1125 +/− 509¹ | 163 +/− 43¹ |
| transwells | | | |
| Activated γδ T cells + | 493 +/− 269² | 261 +/− 150² | 66 +/− 19² |
| anti-TNF Ab | | | |
| Activated γδ T cells + | 1058 +/− 530 | 533 +/− 439 | 144 +/− 30 |
| anti-IFNγ Ab | | | |

1-64. (Canceled).

65. A method for the differentiation and/or maturation of immature myeloid dendritic cells (DC) into HLA-DR, CD86, CD83 and IL12 (p40) dendritic cells comprising incubating said DC with δγ T cells.

66. The method according to claim 65, wherein said immature myeloid DC are derived from monocytes through cytokine treatment chosen from IL-4/GM-CSF or IFN-β/IL-3 or functional analogues thereof.

67. The method according to claim 66, wherein said cytokines are added simultaneously, sequentially or separately with the δγ T cells to the monocytes.

68. The method according to claim 65, wherein said δγ T cells are activated.

69. The method according to claim 68, wherein said activated δγ T cells are produced by treating δγ T cells with an activating agent chosen from microbial products or derivatives thereof.

70. The method according to claim 69, wherein said activating agent is bromohydrin pyrophosphate (BrHPP).

71. The method according to claim 70 wherein BrHPP is present at a concentration of between 10 and 1000 nM.

72. The method according to claim 70 wherein BrHPP is present at a concentration of 200 nM.

73. A method for obtaining a population of HLA-DR, CD86, CD83 and IL12 (p40) dendritic cells comprising the steps of:
(a) isolating monocytes from a patient,
(b) incubating said monocytes in the presence of IFN-β/IL-3, GM-CSF/IL-4 or functional analogues thereof, producing a population of immature myeloid dendritic cells,
(c) isolating δγ T cells, and,
(d) contacting immature myeloid dendritic cells of step (b) with T cells of step (c), whereby said contact is performed directly or indirectly.

74. The method according to claim 73, wherein the contacting of step (d) is performed for 24 hours.

75. The method according to claim 73, wherein said δγ T cells of step (c) are cultured with microbial products or derivatives thereof to activate said δγ T cells prior to contacting said δγ T cells with said immature dendritic cells.

76. The method according to claim 73, further comprising the step of:
(e) presenting a peptide on the surface of said dendritic cells.

77. The method according to claim 75, further comprising the step of:
(e) presenting a peptide on the surface of said dendritic cells.
